(19) United States
(12) Patent Application Publication
(10) Pub. No.: US 2010/0273147 A1
(43) Pub. Date: Oct. 28, 2010

(54) Medical diagnostic system and methods

(76) Inventors: Samuel R. Valenti, New Hope, PA (US); Benigno A. Janeiro, Burlington, NJ (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 11/655,398

(22) Filed: Jan. 19, 2007

Related U.S. Application Data
(60) Provisional application No. 60/760,271, filed on Jan. 19, 2006.

(51) Int. Cl. C12Q 1/68; C12M 1/34; C40B 40/00

(52) U.S. Cl. 435/6; 435/287.2; 506/13

(57) ABSTRACT

A system and methods are provided for a medical diagnostic system that incorporates a genetically encoded digital signature to authenticate a patient. In an illustrative implementation, a platform is provided performing one or more functions including but not limited to patient authentication, diagnostics, transmission, and storage of data in a centralized secure database capable of being accessed by healthcare professionals. In the illustrative implementation, the exemplary medical diagnostic system can comprise a genetic material collector/analyzer. Responsive to inputting genetic material in the collector/analyzer, the collector/analyzer generates a unique genetic-based electronic signature representative of the genetic material. The unique genetic-base electronic signature can then be processed by cooperating parties to authenticate the person providing the genetic sample. In the illustrative operation, such comparison can be accomplished by comparing the generated unique genetic-based electronic signature with a stored genetic-based electronic signature as part of a patient authentication process.

### CLAIM OF PRIORITY AND CROSS-REFERENCE

This non-provisional patent application claims priority to and the benefit of U.S. provisional patent application, 60/760,271, filed on Jan. 19, 2006, entitled, “MEDICAL DIAGNOSTIC SYSTEM AND METHODS” which is herein incorporated by reference in its entirety.

### TECHNOLOGY FIELD

The herein described system and methods relate to a diagnostic system that uses a genetic signature to authenticate users and transmit diagnostic results electronically over a secure data communications network.

### BACKGROUND

The proliferation of communications technologies has led to advancements in various industries ranging from banking to healthcare. Such technologies allow for the dissemination of data between geographic disparately locations for the benefit of the communicating parties. Additionally, such technologies have lead to the development of platforms and applications for use by participating users to allow for remote monitoring and feedback. The remote monitoring applications are used across various industries including information technology, geology, and healthcare. In the healthcare context, such remote monitoring platforms and applications allow patients to be remotely monitored by their healthcare provider as part of patient treatment and follow up.

Current healthcare monitoring/feedback platforms and applications are varied and plenty. For example, with current solutions and approaches provide a health monitoring and diagnostic device (e.g., hand-held device) operable for determining blood lipid levels from test-strip analyses. Such information can then be communicated to healthcare providers to remotely monitor a patient's condition (e.g., diabetes). Such platform can operate to authenticate the patient using a personal identification number (PIN). In addition, this current system can display corresponding diagnostic results, store patient data on a secure patient-held data carrier. Additionally, a secure network-based health assessment and medical records maintenance system is provided for receiving medical information transmitted from the health monitoring and diagnostic device. The system is further described as including integrated communications between the patient, diagnostic device, physician, and pharmacy.

Further, current approaches provide for an analyte detection device (“ADD”) and system for detecting analytes in biological samples. The data acquisition and handling may be performed by employing charged coupled device (CCD) detectors configured to measure white light, ultraviolet light or fluorescence. Such platform can generate data that can be transmitted over a computer network to a medical expert who may then transmit a prescription to the ADD and to a client computer system at a cooperating pharmacy that can operate to fill the prescription.

Additionally, current solutions provide for an integrated system in which biological sample characterization of participating patients can be achieved using one or more sample data collection devices (e.g., a testing kit), or sensors. In operation, the system processes test information for transmission from the collection device to a centralized remote data analyzer via a network (e.g., wireless, Internet, intranet, modem, Ethernet, etc.). Additionally, this current system provides a means for integrating remote patient ID/biometrics (pulse, body temperature, heart rate, etc.), remote diagnostics, insurance, remote physician consultation, pharmacy/pharmacists, and prescriptions.

Current solutions further provide a diagnostic device system, process, and software arrangement for providing a medical dosage recommendation via transmission of biological test results obtained from diagnostic/biosensing devices, and to remote web access processing mechanism. In operation, with such system, remote web access processing operations allow participating patients, physicians, laboratories, insurance companies and/or pharmacies to communicate with each other as part of the providing treatment to patients.

Additionally, current approaches provide a medical data collection kit for use by a patient to collect biological data for subsequent transmission to a remote physician.

Further, current approaches provide an integrated health care system employing biosensors. With this solution, biosensors are capable of generating signals (e.g., analyte measurements) relating to the health of the user. These signals can then be processed and transmitted as needed to various remote destinations. A participating user can selectively control the transmission of biosensor test results to a remotely located physician, insurance provider, or other third party for subsequent review. Additionally, recommendations regarding medication can be made by an insurer that can result in an order placed to a pharmacist or other service providers to prepare materials required for care of a patient (e.g., drugs or other medical aids/services). These patents do not describe the use of a genetically encoded digital signature as a means of identifying and authenticating a patient using the interactive diagnostic system.

Other current solutions provide a system for detecting many types of diseases and other health related issues using self-administered tests and that provides automatic test tracing analysis and reporting. With this solution, during operation, a test kit can be utilized by a patient for collecting, analyzing, and transmitting biological sample test results to a remote location (e.g., physician's office) for subsequent recommendation of prescription medications and dosages appropriate for the patient. The system additionally provides a means for patient identification by utilizing magnetic date/time stamping, and patient provided biometric samples that can comprise a portion of a collected biological sample.

Additionally, current practices provide a method and apparatus for identifying a biological sample obtained wherein the analysis results obtained is associated with a DNA fingerprint.

However current solutions fall short to provide a secure, trusted authentication and verification operation that provides absolute confidence to the healthcare provider that the remote monitoring and treatment is directed to the desired patient (e.g., participating user).

It is appreciated that there exists a need for system and methods to overcome the shortcomings of existing solutions and approaches. Specifically, system and methods directed to a reliable and robust authentication procedure such as genetically encoded digital signatures for authenticating patients, individuals and transmitting the data over networks as part of remote healthcare monitoring and treatment platforms.

### SUMMARY

A system and methods are provided for a medical diagnostic system that incorporates a genetically encoded digital signature to authenticate a patient. In an illustrative implementation, a platform can perform one or more functions including but not limited to patient authentication and identification, diagnostics, transmission, and storage of data in a centralized secure database capable of being accessed by healthcare professionals. In the illustrative implementation, the herein described system and methods can comprise protocols to comply with one or more regulatory agency rule and/or regulation. In the illustrative implementation, the exemplary medical diagnostic system can comprise a genetic material collector/analyzer electronically coupled to a computing environment.

In the illustrative operation, a patient can provide a bodily fluid sample for placement in the genetic material collector/analyzer for processing. Responsive to such input, the collector/analyzer generates a unique genetic-based electronic signature representative of the genetic material. The unique genetic-base electronic signature can then be processed by cooperating parties to authenticate the person providing the genetic sample. In the illustrative operation, such comparison can be accomplished by comparing the generated unique genetic-based electronic signature with a stored genetic-based electronic signature as part of a patient authentication process.

In the illustrative operation, the medical diagnostic system can be used by healthcare professional as a means for remotely diagnosing ailments, prescribing therapies and using the digital signature in combination with clinical trial data to pre-screen patients for appropriate therapies and thus customize patient care in addition to monitoring therapy efficacy and compliance. In the illustrative operation, insurance providers can make use of the diagnostic system to monitor compliance and adjust insurance premiums based on patient risk levels due to compliance and non-compliance.

Other illustrative uses of the herein described system and methods can include but are not limited to: clinical trials of drugs and bio-technology products as part of test-patient compliance protocols as well as to biologically monitor and confirm side effects of such drug/bio-tech trial; law enforcement and employers can use the system as a means to monitor and screen for alcohol and drug abuse; and the incorporation of a digital signature into an exemplary diagnostic devices can provide a universal, indisputable form of identification, which, in an illustrative implementation, can be used by a health care professional to remotely authenticate a patient's identity.

Other features of the herein described systems and methods are described below.

### BRIEF DESCRIPTION OF THE DRAWINGS

The medical diagnostic system and methods are further described with reference to the accompanying drawings in which:

FIG. 1 is a block diagram of an exemplary computing environment in accordance with an implementation of the herein described systems and methods;

FIG. 2 is a block diagram of an exemplary networked computing environment;

FIG. 3 is a block diagram showing the cooperation of exemplary components of another illustrative implementation in accordance with the herein described systems and methods; and

FIG. 4 is a flow diagram of the processing performed in an illustrative operation in accordance with the herein described systems and methods.

### DETAILED DESCRIPTION

### Overview:

The herein described system and methods provide a diagnostic system that is capable of establishing a patient's genetic signature, authenticating the genetic signature, collecting and analyzing diagnostic data, securing and electronically transmitting the data over a network, and utilizing data results as the primary means for making recommendations. The exemplary diagnostic system will provide healthcare providers with a universal foolproof patient identification signature, a means to diagnose an ailment and prescribe medication and to prompt insurance companies for payment of services provided. In an illustrative operation, the system can be used as a means to monitor patient compliance to a prescribed medication regimen, to identify potential drug interactions, and to monitor patient compliance during clinical study trials. The genetic signature can also be used to identify a therapy's efficacy and potential adverse side effects by scanning clinical trial data for known genetic predispositions based on the patient's DNA signature.

Additionally, the exemplary diagnostic system can have applications in various areas including but not limited to: law enforcement for monitoring drug and alcohol use in addition to being used by employers to screen workers undertaking dangerous jobs. Health insurance companies can also use the herein described systems and methods to monitor patient's compliance and based on the results, adjust the patient's risk factor and hence insurance premiums. In an illustrative implementation, the genetic signature can be a universal code that can be easily implemented as a worldwide platform and used in combination with diagnostic devices.

### Illustrative Computing Environment:

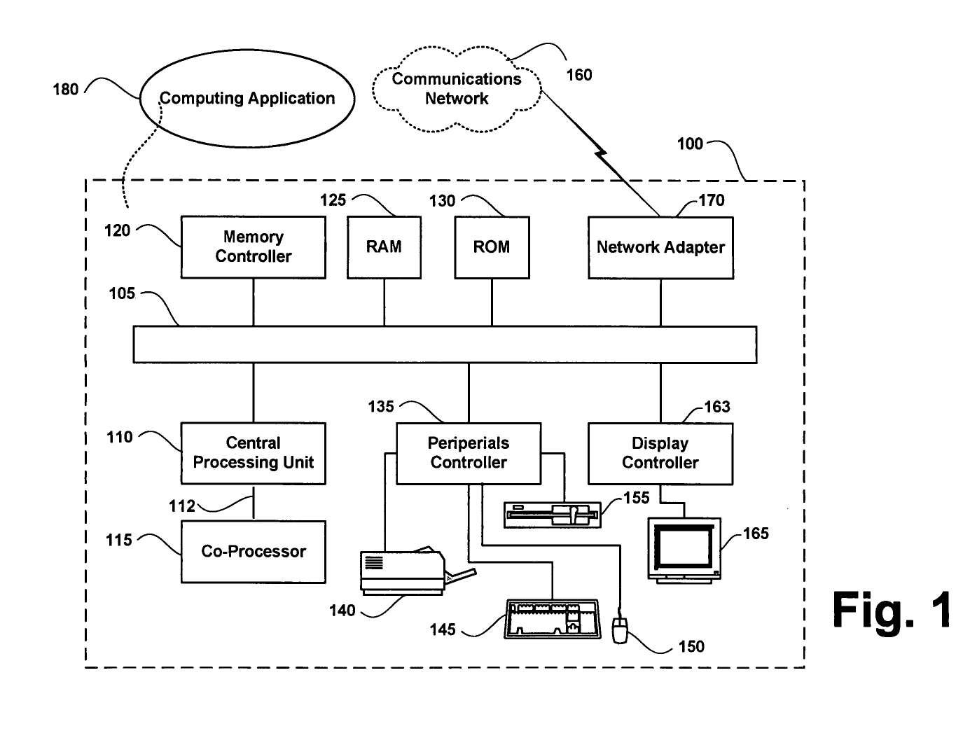

FIG. 1 depicts an exemplary computing system 100 in accordance with herein described system and methods. The computing system 100 is capable of executing a variety of computing applications 180. Computing application 180 can comprise a computing application, a computing applet, a computing program and other instruction set operative on computing system 100 to perform at least one function, operation, and/or procedure. Exemplary computing system 100 is controlled primarily by computer readable instructions, which may be in the form of software. The computer readable instructions can contain instructions for computing system 100 for storing and accessing the computer readable instructions themselves. Such software may be executed within central processing unit (CPU) 110 to cause the computing system 100 to do work. In many known computer servers, workstations and personal computers CPU 110 is implemented by micro-electronic chips CPUs called microprocessors. A coprocessor 115 is an optional processor, distinct from the main CPU 110 that performs additional functions or assists the CPU 110. The CPU 110 may be connected to co-processor 115 through interconnect 112. One common type of coprocessor is the floating-point coprocessor, also called a numeric or math coprocessor, which is designed to perform numeric calculations faster and better than the general-purpose CPU 110.

In operation, the CPU 110 fetches, decodes, and executes instructions, and transfers information to and from other resources via the computer's main data-transfer path, system bus 105. Such a system bus connects the components in the computing system 100 and defines the medium for data exchange. Memory devices coupled to the system bus 105 include random access memory (RAM) 125 and read only memory (ROM) 130. Such memories include circuitry that allows information to be stored and retrieved. The ROMs 130 generally contain stored data that cannot be modified. Data stored in the RAM 125 can be read or changed by CPU 110 or other hardware devices. Access to the RAM 125 and/or ROM 130 may be controlled by memory controller 120. The memory controller 120 may provide an address translation function that translates virtual addresses into physical addresses as instructions are executed.

In addition, the computing system 100 can contain peripherals controller 135 responsible for communicating instructions from the CPU 110 to peripherals, such as, printer 140, keyboard 145, mouse 150, and data storage drive 155. Display 165, which is controlled by a display controller 163, is used to display visual output generated by the computing system 100. Such visual output may include text, graphics, animated graphics, and video. The display controller 163 includes electronic components required to generate a video signal that is sent to display 165. Further, the computing system 100 can contain network adaptor 170 which may be used to connect the computing system 100 to an external communication network 160.

### Illustrative Computer Network Environment:

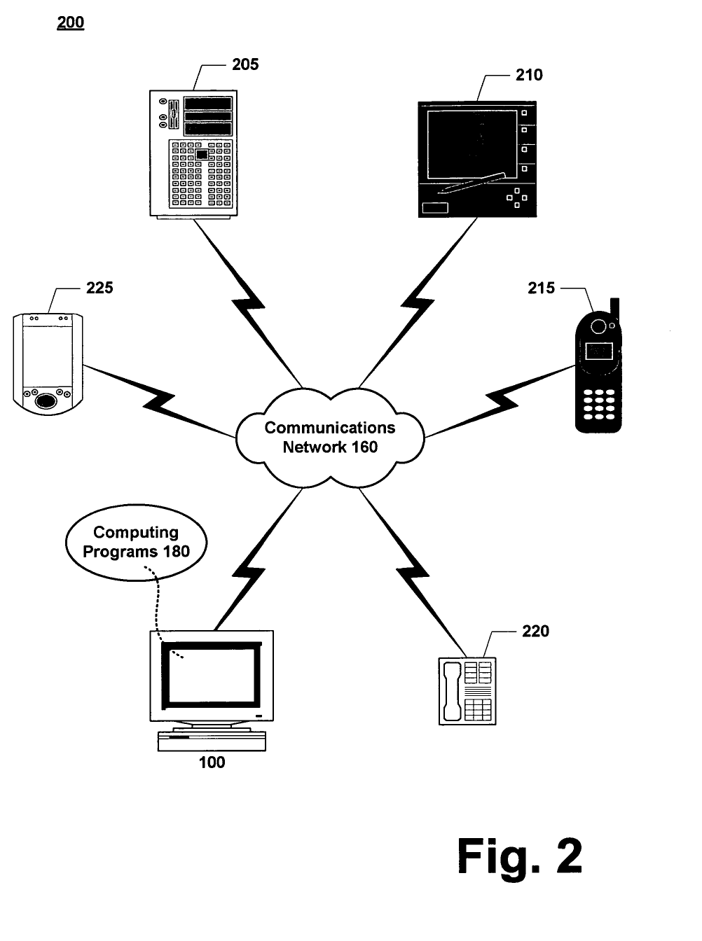

Computing system 100, described above, can be deployed as part of a computer network. In general, the above description for computing environments applies to both server computers and client computers deployed in a network environment. FIG. 2 illustrates an exemplary illustrative networked computing environment 200, with a server in communication with client computers via a communications network, in which the herein described apparatus and methods may be employed. As shown in FIG. 2, server 205 may be interconnected via a communications network 160 (which may be either of, or a combination of a fixed-wire or wireless LAN, WAN, intranet, extranet, peer-to-peer network, the Internet, or other communications network) with a number of client computing environments such as tablet personal computer 210, mobile telephone 215, telephone 220, personal computer 100, and personal digital assistance 225. In a network environment in which the communications network 160 is the Internet, for example, server 205 can be dedicated computing environment servers operable to process and communicate data to and from client computing environments 100, 210, 215, 220, and 225 via any of a number of known protocols, such as, hypertext transfer protocol (HTTP), file transfer protocol (FTP), simple object access protocol (SOAP), or wireless application protocol (WAP). Each client computing environment 100, 210, 215, 220, and 225 can be equipped with browser operating system 180 operable to support one or more computing applications such as a web browser (not shown), or a mobile desktop environment (not shown) to gain access to server computing environment 205. Client computing environments 100, 210, 215, 200, and 225 can operate to execute one or more computing applications and applets operating to process one or more high level computing language (e.g., HTML, JAVA, FLASH Media, etc.).

In operation, a user (not shown) may interact with a computing application running on a client computing environments to obtain desired data and/or computing applications. The data and/or computing applications may be stored on server computing environment 205 and communicated to cooperating users through client computing environments 100, 210, 215, 220, and 225, over exemplary communications network 160. A participating user may request access to specific data and applications housed in whole or in part on server computing environment 205. These data may be communicated between client computing environments 100, 210, 215, 220, and 220 and server computing environments for processing and storage. Server computing environment 205 may host computing applications, processes and applets for the generation, authentication, encryption, and communication of web services and may cooperate with other server computing environments (not shown), third party service providers (not shown), network attached storage (NAS) and storage area networks (SAN) to realize such web services transactions.

### Genetic Sample Processing

Within the healthcare system there are numerous components capable of generating data that employ differing technologies that, generally, also include some level of error. Such errors can be attributed to the use of different assay formats, different supplier technologies, different researchers and experimental conditions. When all of these sources are added together, a resulting database using such instrumentalities to collect data can become fraught with inconsistencies.

The standard platform of herein described system and methods illustratively provides a direct authentication process that, illustratively, can leverage a combination of micro-fluidics and micro-array diagnostic methods, provide a direct detection method, provide protocols for authentication, transmission and storage of information to a central secure database to reduce and/or eliminate the errors caused by inconsistencies created by current solutions.

There are millions of base pairs in each person's DNA and every individual has a different sequence. By using these sequences individuals can be identified solely by the sequence of their base pairs. There are millions of base pairs and scientists are able to use techniques that have been developed to identify shorter repeating patterns in DNA.

These patterns do not reveal an individual's “fingerprint,” but they are able to determine whether two DNA samples are from the same person. With current practices, scientists can use a small number of sequences of DNA that are known to vary among individuals significantly, and analyze such sequences to get a certain probability of a match. Such processing is described in detail in, “DNA Fingerprinting in Human Health and Society,” Betsch, David, Biotechnology Training Programs, Inc., Iowa State University Office of Biotechnology, which is herein incorporated by reference in its entirety.

Similar to fingerprints that came into use by law enforcement in the 1930s, each person has a unique DNA fingerprint, however, a conventional fingerprint can be altered by surgery, and a DNA fingerprint is the same for every cell, tissue, and organ of a person and, as such, cannot be altered by any known treatment. DNA fingerprinting has established a powerful method for identifying and distinguishing individuals. For example, government law enforcement agencies (e.g., federal bureau of investigation (FBI)) an police labs around the U.S. and the world have begun to use DNA fingerprints to link suspects to biological evidence—blood or semen stains, hair, or items of clothing—found at the scene of a crime.

Since 1987, hundreds of cases have been decided with the assistance of DNA fingerprint evidence. In these applications, DNA fingerprints bring and unprecedented, nearly perfect accuracy to the determination. Since every organ or tissue of an individual contains the same DNA fingerprint, the collection of DNA fingerprints from all personnel are being collected by various enterprises (e.g., U.S. armed services) for use later, in case they are needed to identify casualties or persons missing in action. Such DNA method is appreciated to be superior to conventional identification methodologies and instrumentalities including the dogtags, dental records, and blood typing strategies currently in use.

Conventional DNA fingerprinting in a laboratory can be accomplished by the following: (1) DNA isolation using biological samples such as blood, hair, saliva, or skin; (2) cutting, sizing, and sorting restriction enzymes for use in cutting DNA at selected locations (e.g., the enzyme EcoR1, found in bacteria, operates to cut DNA only when the sequence GAATTC occurs. The DNA pieces can be sorted according to size by a sieving technique called electrophoresis. Also, the DNA pieces can be passed through a gel made from seaweed agarose (a jelly-like product made from seaweed); (3): Transfer of DNA to nylon; (4-5) Probing and adding radioactive or colored probes to the nylon sheet produces a pattern called the DNA fingerprint; (6) The final DNA fingerprint is built by using several probes (5-10 or more) simultaneously (e.g., the end DNA fingerprint can resemble the bar codes used by grocery store scanners).

Current DNA technologies used in forensic investigations include, Restriction Fragment Length Polymorphism (RFLP), PCR Analysis, STR Analysis, Mitochondrial DNA Analysis, Y-Chromosome Analysis, and VNTRs.

RFLP, Restriction Fragment Length Polymorphism (RFLP), is a technique for analyzing the variable lengths of DNA fragments that result from digesting a DNA sample using a selected enzyme. This enzyme, which generally is a restriction endonuclease, can operate to cut DNA at a specific sequence pattern know as a restriction endonuclease recognition site. The presence or absence of certain recognition sites in a DNA sample can generate variable lengths of DNA fragments, which can be separated using gel electrophoresis. They are then hybridized with DNA probes that operate to bind to a complementary DNA sequence in the sample. RFLP is one of the original applications of DNA analysis to forensic investigation. With the development of newer, more efficient DNA-analysis techniques, RFLP is not used as much as it once was because it requires relatively large amounts of DNA. In addition, samples degraded by environmental factors, such as dirt or mold, do not work well with RFLP.

PCR Analysis (polymerase chain reaction) can be used to make millions of exact copies of DNA from a biological sample. DNA amplification with PCR allows DNA analysis on biological samples as small as a few skin cells. With RFLP, DNA samples would have to be about the size of a quarter. The ability of PCR to amplify such tiny quantities of DNA enables even highly degraded samples to be analyzed. Great care, however, must be taken to prevent contamination with other biological materials during the identifying, collecting, and preserving of a sample. STR Analysis, Short tandem repeat (STR), technology is used to evaluate specific regions (loci) within nuclear DNA. Variability in STR regions can be used to distinguish one DNA profile from another.

For example, the Federal Bureau of Investigation (FBI) uses a standard set of 13 specific STR regions for CODIS. CODIS is a software program that operates local, state, and national databases of DNA profiles from convicted offenders, unsolved crime scene evidence, and missing persons. The odds that two individuals will have the same 13-loci DNA profile are about one in one billion.

Mitochondrial DNA Analysis (mtDNA) can be used to examine the DNA from samples that cannot be analyzed by RFLP or STR. Nuclear DNA is extracted from samples for use in RFLP, PCR, and STR; however, mtDNA analysis uses DNA extracted from another cellular organelle called a mitochondrion. While older biological samples that lack nucleated cellular material, such as hair, bones, and teeth, cannot be analyzed with STR and RFLP, they can be analyzed with mtDNA. In the investigation of cases that have gone unsolved for many years, mtDNA is extremely valuable.

For example, mothers have the same mitochondrial DNA as their daughters. This is because the mitochondria of each new embryo come from the mother's egg cell. The father's sperm contributes only nuclear DNA. Comparing the mtDNA profile of unidentified remains with the profile of a potential maternal relative can be an important technique in missing person investigations. Y-Chromosome Analysis passed directly from father to son, so the analysis of genetic markers on the Y chromosome is especially useful for tracing relationships among males or for analyzing biological evidence involving multiple male contributors.

Strands of DNA have pieces that contain genetic information which informs an organism's development (exons) and pieces that, apparently, supply no relevant genetic information at all (introns). Although the introns may seem useless, it has been found that they contain repeated sequences of base pairs. These sequences, called Variable Number Tandem Repeats (VNTRs), can contain anywhere from twenty to one hundred base pairs. Every human being has some VNTRs. To determine if a person has a particular VNTR, a Southern Blot is performed, and then the Southern Blot is probed, through a hybridization reaction, with a radioactive version of the VNTR in question. The pattern, which results from this process, is what is often referred to as a DNA fingerprint. Because VNTR patterns are inherited genetically, a given person's VNTR pattern is unique. The more VNTR probes used to analyze a person's VNTR pattern, the more distinctive and individualized that pattern, or DNA fingerprint, will be.

As described, Southern Blot is one way to analyze the genetic patterns, which appear in a person's DNA. Performing a Southern Blot generally involves:

(1) Isolating the DNA in question from the rest of the cellular material in the nucleus. This can be done either chemically, by using a detergent to wash the extra material from the DNA, or mechanically, by applying a large amount of pressure in order to “squeeze out” the DNA.

(2) Cutting the DNA into several pieces of different sizes. This is done using one or more restriction enzymes.

(3) Sorting the DNA pieces by size. The process by which the size separation, “size fractionation,” is done is called gel electrophoresis. The DNA is poured into a gel, such as agarose, and an electrical charge is applied to the gel, with the positive charge at the bottom and the negative charge at the top. Because DNA has a slightly negative charge, the pieces of DNA will be attracted towards the bottom of the gel; the smaller pieces, however, will be able to move more quickly and thus further towards the bottom than the larger pieces. The different-sized pieces of DNA will therefore be separated by size, with the smaller pieces towards the bottom and the larger pieces towards the top.

(4) Denaturing the DNA, so that the entire DNA is rendered single-stranded. This can be done either by heating or by chemically treating the DNA in the gel.

(5) Blotting the DNA. The gel with the size-fractionated DNA is applied to a sheet of nitrocellulose paper, and then baked to permanently attach the DNA to the sheet. The Southern Blot is now ready to be analyzed.

In analyzing a Southern Blot, a radioactive genetic probe is used in a hybridization reaction with the DNA in question. If an X-ray is taken of the Southern Blot after a radioactive probe has been allowed to bond with the denatured DNA on the paper, only the areas where the radioactive probe binds will show up on the film. This allows researchers to identify, in a particular person's DNA, the occurrence and frequency of the particular genetic pattern contained in the probe.

Hybridization Reaction is the binding, of two genetic sequences. The binding occurs because of the hydrogen bonds between base pairs. When making use of hybridization in the laboratory, DNA must first be denatured, usually by using heat or chemicals. Denaturing is a process by which the hydrogen bonds of the original double-stranded DNA are broken, leaving a single strand of DNA whose bases are available for hydrogen bonding. Once the DNA has been denatured, a single-stranded radioactive probe can be used to see if the denatured DNA contains a sequence similar to that on the probe. The denatured DNA is put into a plastic bag along with the probe and some saline liquid; the bag is then shaken to allow sloshing. If the probe finds a fit, it will bind to the DNA.

DNA fingerprinting can be used to diagnose inherited disorders, which may include cystic fibrosis, hemophilia, Huntington's disease, familial Alzheimer's, sickle cell anemia, thalassemia, and many others.

Early detection of such disorders enables individuals and healthcare professionals to prepare for proper treatment.

Generally, diseases have a genetic component, whether inherited or resulting from the body's response to environmental stresses like viruses or toxins. The ultimate goal is to use the genetic identified information to detect thousands of diseases that afflict humankind. Nevertheless, the road from gene identification to effective detection is long and fraught with challenges. In the meantime, biotechnology companies are racing ahead with commercialization by designing diagnostic tests to detect errant genes in people suspected of having particular diseases or of being at risk for developing them. Additionally, an increasing number of gene tests are becoming available commercially.

The routine use of blood samples is on the decline. Buccal swabs are rapidly becoming the DNA specimen of choice. The DNA testing specimen is collected by gently rubbing the cheeks inside the mouth with long swabs similar to Q-tips. The buccal cells that come off in the process require no refrigeration or preservatives and do not need immediate shipping to the paternity testing laboratory. The DNA tests utilized in laboratories are the same DNA tests as used when testing blood for DNA identification of specimens. Buccal swab DNA specimens are not affected by bacteria, toothpaste, chewing tobacco or other tobacco products, lipstick, or nursing (mother's milk). Bacterial DNA does not affect the testing, as bacteria do not contain the DNA sequences examined in the test. No fasting is required prior is specimen collection.

Buccal swabs is a non-invasive DNA specimen collection procedure uses four cotton swabs that are similar to ordinary Q-tips to collect epithelial cells by stroking the lining of the inner cheek. These cells contain the DNA required to perform parentage testing. The testing procedures utilized on DNA extracted from buccal cells are the same procedures as performed on DNA extracted from white blood cells.

The some of the advantages provided by a buccal swab collection procedure are: self administered non-invasive procedure, little or no biohazardous waste, procedure permits specimen retention for future testing, no age restriction, reduces the risk of testing of HIV drug abusers, no additional cost for buccal swab specimen collection and processing.

Scientists are fine-tuning a test that replaces blood tests with saliva samples to diagnose disease. For example, molecules found in the blood or urine can also be found somewhere in the oral cavity and a sensitive enough test can be developed to measure most things in the oral cavity. There is a saliva test to diagnose in a matter of minutes if an individual has been infected with the HIV virus. Also, a saliva test that can detect oral cancer is currently being developed. Once targets have been identified, saliva can be used for disease diagnostics. Individuals have about 3,000 copies of RNA in their saliva and there are cores of RNA, 185 of them, that are common in all normal individuals; four RNA markers out of the 3,000 present in all normal individuals can be used to screen for cancer. The advances in microfluidics and microarray technologies have allowed for the development of tests that are sensitive enough to even detect a single virus or bacteria, thus, allowing the development of devices for diagnostic testing.

### Exemplary Medical Diagnostic Environment:

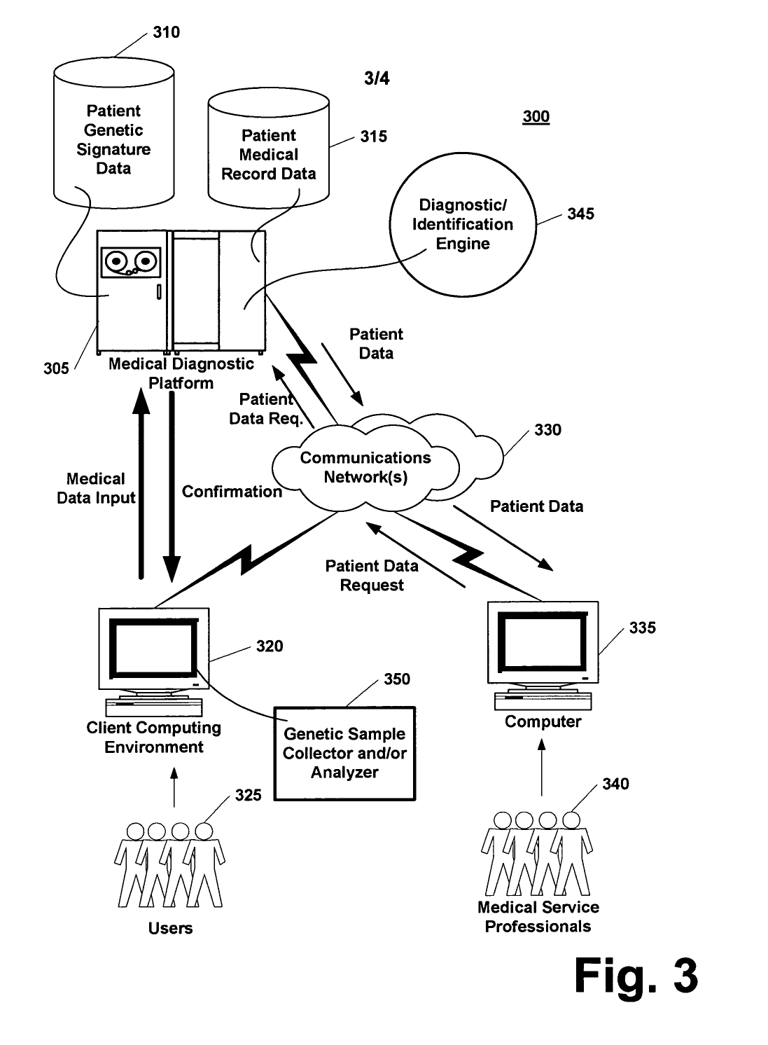

FIG. 3 shows exemplary medical diagnostic environment 300. As is shown in FIG. 3, medical diagnostic environment 300 comprises medical diagnostic platform 305, communications network 330, client computing environment 320 coupled to genetic sample collector/analyzer, diagnostic/identification engine 345, and computer 335. Further, as is shown in FIG. 3, medical diagnostic platform 305 can be electronically coupled to patient genetic signature data store 310 and patient medical record data 315.

In an illustrative operation, users 325 (e.g., patients) can interface with genetic sample collector/analyzer 350 to provide a genetic material sample (e.g., bodily fluid). Responsive to the genetic input, genetic sample collector/analyzer 350 can process the genetic sample (e.g., by cooperating with an application executing on client computing environment 320 and/or by cooperating with diagnostic/identification engine 345) to generate a unique genetic-based electronic signature. In the illustrative operation, the generated unique genetic-based electronic signature can be communicated to the medical diagnostic platform 305 for processing and storage (e.g., storage on patient genetic signature data store 310) and can be associated with patient medical record data on patient medical record data store 315 using communications network 330. Additionally, in the illustrative operation, medical service professionals 340 can interface with computer 335 to obtain patient medical record information from patient medical record data store 315 using communications network 330.

Additionally, in an illustrative implementation, diagnostic/identification engine 345 can comprise a computing application providing one or more instructions to medical diagnostic platform 305 to process data received from client computing environment 320 according to one or more selected diagnostic/identification paradigms (not shown) to generate patient genetic signature data (not shown) for storage in patient genetic signature data store 310.

In an illustrative implementation, exemplary medical diagnostic system 300 can integrate DNA sequencing technologies and data encryption in an effort to adhere to governmental agency (e.g., Food and Drug Administration—FDA) communication protocols for transmission of personal data.

In an illustrative operation, an individual's saliva or blood sample can be used to extract a DNA sample. This sample can the be used to create a DNA sequence profile that is unique to the individual whom provided the sample. This DNA sequence can then be digitized to create a DNA fingerprint that becomes the individual's reference signature as stored in a centralized secure network.

In another illustrative operation, medical diagnostic system 300 can comprise an exemplary DNA sequencer that is equipped with software to be able to process data collected and generated by the DNA sequencer to match against a reference DNA signature. Once a reference DNA signature has been identified, an encrypted file can be created with an individuals DNA signature and diagnostic test results. This file is then sent to an exemplary centralized network (as shown in FIG. 2) for secure storage.

In another illustrative implementation, a handheld DNA sequencer (not shown) having wireless communication capabilities can be used by hospitals and emergency medical technicians (EMT) as part of field operations (e.g., responding to a emergency call; trauma care, etc.). This handheld device can be used to collect a DNA sequence data from patients and victims in need of emergency care and match them against a reference database (not shown). The DNA signature could be used to quickly identify patients or victims, create a profile or case, retrieve medical information and insurance data and notify family members.

In another illustrative implementation, exemplary medical diagnostic system 300 can comprise a medical diagnostic too (analyzer 350) that can operate to identify the causal agent of a common non-life threatening disease (e.g. flu, cold) while allowing for individual identification of the user. The results of an individualized diagnostic test performed remotely (away from a doctor's office or a diagnostic lab) can be transmitted to a medical center where a doctor can in turn prescribe an appropriate medicine to cure the disease. Medical prescription can be sent via Internet or telephone to the patient and/or the pharmacist, allowing home or office delivery of a drug as appropriate (not shown).

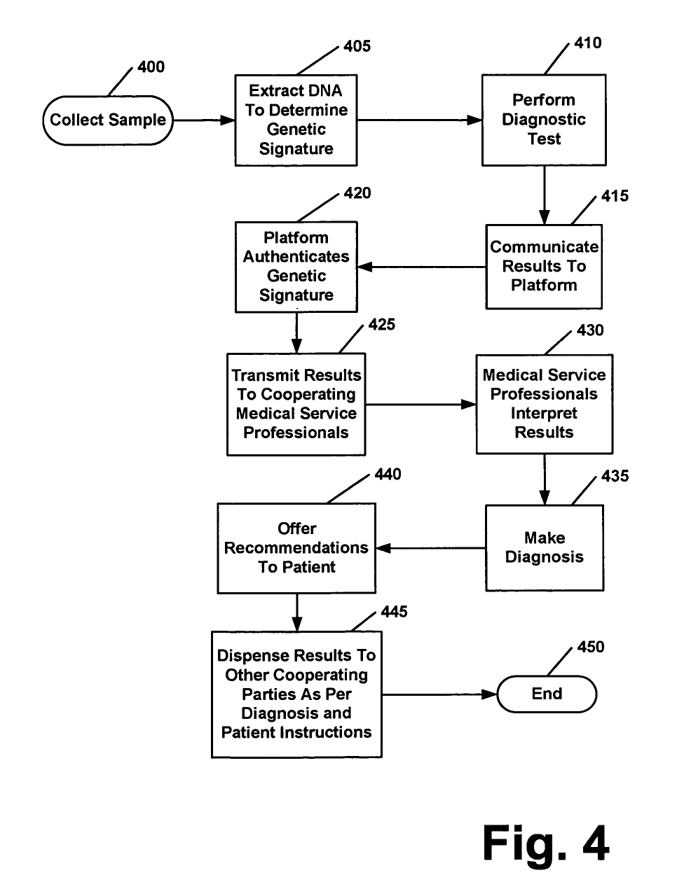

FIG. 4 shows exemplary processing performed by exemplary medical diagnostic system 305 of FIG. 3. As is shown in FIG. 4, processing begins at block 400 where a genetic sample is collected. DNA is then extracted at block 405 to generate a unique genetic-based electronic signature. Processing then proceeds to block 410 where one or more diagnostic tests can be performed on the genetic sample. Processing then proceeds to block 415 where the data is communicated to the platform. The platform can then authenticate the genetic signature at block 420 and transmit the results to cooperating medical service professionals at block 425. The medical service professionals can then interpret the results at block 430 so that they can make a diagnosis at block 435 and offer recommendations for treatment at block 440. From there the results of the medical diagnosis can be communicated to other cooperating parties (e.g., pharmacist, pharmaceutical company, physician, etc.) for use as part of patient care and/or monitoring. Processing then terminates at block 450.

It is understood that the herein described systems and methods are susceptible to various modifications and alternative constructions. There is no intention to limit the invention to the specific constructions described herein. On the contrary, the invention is intended to cover all modifications, alternative constructions, and equivalents falling within the scope and spirit of the invention.

It should also be noted that the herein described systems and methods may be implemented in a variety of computer environments (including both non-wireless and wireless computer environments), partial computing environments, and real world environments. The various techniques described herein may be implemented in hardware or software, or a combination of both. Preferably, the techniques are implemented in computing environments maintaining programmable computers that include a processor, a storage medium readable by the processor (including volatile and non-volatile memory and/or storage elements), at least one input device, and at least one output device. Computing hardware logic cooperating with various instructions sets are applied to data to perform the functions described above and to generate output information. The output information is applied to one or more output devices. Programs used by the exemplary computing hardware may be preferably implemented in various programming languages, including high level procedural or object oriented programming language to communicate with a computer system. Illustratively the herein described apparatus and methods may be implemented in assembly or machine language, if desired. In any case, the language may be a compiled or interpreted language. Each such computer program is preferably stored on a storage medium or device (e.g., ROM or magnetic disk) that is readable by a general or special purpose programmable computer for configuring and operating the computer when the storage medium or device is read by the computer to perform the procedures described above. The apparatus may also be considered to be implemented as a computer-readable storage medium, configured with a computer program, where the storage medium so configured causes a computer to operate in a specific and predefined manner.

Although an exemplary implementation of the herein described system and methods have been described in detail above, those skilled in the art will readily appreciate that many additional modifications are possible in the exemplary embodiments without materially departing from the novel teachings and advantages of the herein described system and methods. Accordingly, these and all such modifications are intended to be included within the scope of this herein described system and methods. The herein described system and methods may be better defined by the following exemplary claims.

What is claimed is:

1. A medical diagnostic system comprising:
a genetic sample collector and/or analyzer;
a diagnostic/identification engine operatively cooperating with the genetic sample collector and/or analyzer comprising one or more instructions to process genetic sample data according to one or more selected diagnostic/identification paradigms,
wherein the selected diagnostic/identification paradigms comprise genetic fingerprinting.

2. The system as recited in claim 1 further comprising a communications network operatively coupled to the genetic sample collector and/or analyzer for communicating genetic diagnostic/identification data to the diagnostic/identification engine.

3. The system as recited in claim 2 further comprising a computing environment operatively coupled to the genetic sample collector and/or analyzer for use in communicating genetic diagnostic/identification data to the diagnostic/identification engine.

4. The system as recited in claim 1 further comprising a data store operable to store genetic signature data generated by the diagnostic/identification engine and/or genetic sample collector and/or analyzer.

5. The system as recited in claim 4 further comprising a data store operable to store patient medical record data.

6. The system as recited in claim 5 further comprising a computing environment operable by medical service professionals and operatively coupled to the diagnostic/identification engine through a communications network to communicate genetic signature data and/or patient medical records data.

7. The system as recited in claim 1 wherein the diagnostic/identification engine comprises a computing application operable on a computing environment.

8. The system as recited in claim 1 wherein the genetic sample collector and/or analyzer comprises one or more microfluidic arrays.

9. The system as recited in claim 1 wherein the genetic sample collector and/or analyzer comprise a hand-held form factor.

10. The system as recited in claim 1 wherein the genetic sample collector and/or analyzer is operable to communicate data wirelessly.

11. A method for medical diagnosis and/or identification comprising:
collecting a genetic sample;
processing the genetic sample according to one or more selected genetic processing paradigms to generate data representative of a genetic signature and/or genetic diagnostic data;
communicating the generated genetic signature data and/pr genetic diagnostic data for subsequent use as part of identification/diagnosis operations; and
using the generated genetic signature data as part of identifying and/or diagnosing a participating user.

12. The method as recited in claim 11 further comprising generating a DNA fingerprint using the collected genetic sample.

13. The method as recited in claim 11 further comprising communicating data representative of the collected genetic sample to a cooperating identification/diagnosis engine for processing to generate the genetic signature.

14. The method as recited in claim 11 further comprising collecting the genetic sample by a genetic sample collector and/or analyzer.

15. The method as recited in claim 11 further comprising processing the collected genetic sample according to a selected genetic processing paradigm comprising: Restriction Fragment Length Polymorphism (RFLP), PCR Analysis, STR Analysis, Mitochondrial DNA Analysis, Y-Chromosome Analysis, and VNTRs.

16. The method as recited in claim 11 further comprising providing the genetic sample for collection by a participating user.

17. The method as recited in claim 16 further comprising receiving the generated genetic signature data and/or genetic diagnostic data by a cooperating medical service provider for use in identifying and diagnosing the health of the participating user.

18. The method as recited in claim 17 further comprising providing one or more recommendations for treatment to the participating user by the medical service provider based on the received genetic signature and/or genetic diagnostic data.

19. The method as recited in claim 16 further comprising receiving the generated genetic signature data and/or genetic diagnostic data by a cooperating law enforcement personnel for use in identifying the participating user as part of one or more law enforcement activities.

20. The method as recited in claim 11 further comprising storing the generated genetic signature data and/or genetic diagnostic data in a cooperating data store for subsequent use.
